(19) United States
(12) Patent Application Publication
(10) Pub. No.: US 2004/0067527 A1
(43) Pub. Date: Apr. 8, 2004

(54) Intracellular delivery system for protein phosphatases and other polypeptides

(76) Inventors: Sara Lavi, Kiryat Ono (IL); Ronit Satchi-Fainaro, Tel-Aviv (IL)

Correspondence Address: John P White, Cooper & Dunham, 1185 Avenue of the Americas, New York, NY, 10036, US

(21) Appl. No.: 10/343,163

(22) PCT Filed: Jul. 26, 2001
(86) PCT No.: PCT/IL01/00689

(51) Int. Cl. G01N 33/53; C08G 63/48

(52) U.S. Cl. 435/7.1; 525/54.1

(57) ABSTRACT

This invention provides a polymer-based intracellular delivery system for protein phosphatases and other polypeptides. This delivery system can be used to deliver polypeptides for anti-tumor, anti-inflammatory, or immunosuppressive therapy, for treatment of genetic disorder or disease, and for therapy of any condition which requires intracellular delivery of polypeptides. Preferred embodiments according to the invention utilize acrylamide based polymers, most preferably copolymers comprising hydroxypropyl methacrylamide.

### FIELD OF THE INVENTION

The present invention relates to a polymer-based intracellular delivery system for protein phosphatases and other polypeptides, useful for the delivery of polypeptides for anti-tumor, anti-inflammatory, or immunosuppressive therapy, for treatment of genetic disorder or disease, for therapy of any condition which requires intracellular delivery of polypeptides, and for the elucidation of the activity of unknown proteins or polypeptides.

### BACKGROUND OF THE INVENTION

Throughout this specification, various scientific publications are referenced. Full citations for these references may be found at the end of the specification immediately preceding the claims. Additionally, various patent publications are cited in the specification. The disclosure of all these publications in their entireties are hereby incorporated by reference into this specification in order to more fully describe the state of the art to which this invention pertains.

Cancer Therapy

Most anti-tumor agents used clinically act upon metabolic pathways related to cell growth and high mitotic activity. These effects are usually so non-specific that simultaneous serious damage to healthy cells occurs. Tissues with high cellular division rates are particularly affected (bone marrow, intestinal mucosa, the hair follicle cells) leading to unpleasant dose-limiting side effects and decrease in the quality of life.

Lack of selectivity is only one, albeit major, obstacle hindering the optimization of drug effectiveness. Others include inaccessibility of target, premature drug metabolism and allergic reactions (Gregoriadis, 1989).

Chemotherapeutic treatment of neoplastic diseases is often restricted by adverse systemic toxicity which limits the dose of drug that can be administered, or by the appearance of drug resistance. Resistance to a cytostatic/cytotoxic agent can be based on many factors such as premature inactivation leading to insufficient concentration at the target site, formation of inactivating antibodies, increase in the levels of p-glycoprotein that can pump the drug out of the tumor cell, and appearance of DNA repair mechanisms (Mutschler and Derendorf, 1995).

The main conclusion that can be drawn from all these difficulties in achieving effective cancer chemotherapy is that there is a great demand for new anti-tumor drugs that may not have the toxicity and resistance problems described above. There is also a great demand for innovative drug delivery systems that can target anti-tumor drugs in a better manner and that can overcome resistance in its many forms.

Genes have now been identified that are involved in transformation such as Ras, Fos PDGF, erb-B, erb-B2, RET, c-myc, Bcl-2, APC, NF-1, Rb, p53, etc. The genes fall into two broad categories, proto-oncogenes and tumor suppressor genes. Proto-oncogenes code for proteins that stimulate cell division and when mutated (oncogenes) cause stimulatory proteins to be overactive, with the result that cells over-proliferate. Tumor suppressor genes code for proteins that suppress cell division. Mutations and/or aberrant regulation can cause these proteins to be inactivated, thereby rendering the cells without proliferation restraint. Additionally, E2F and p53 and others can act as both oncogene and tumor suppressor gene when improperly expressed. Among the oncogenes and tumor suppressor genes are motifs which act as transcription factors and as protein kinases. The identification of these specific genes has increased our knowledge of the cell life cycle.

Phosphorylation of structural and regulatory proteins including oncogenes and tumor suppressor genes is a major intracellular control mechanism in eukaryotes (Wera and Hemmings, 1995; Cohen, 1989). Protein phosphorylation and dephosphorylation is part of the regulatory cycle for signal transduction, cell cycle progression and transcriptional control. Protein kinases and protein phosphatases both have roles in the phosphorylation/dephosphorylation cycle, respectively. Altered expression of the genes coding for these proteins can lead to failure of protein phosphorylation which can result in tumor formation. For example, Erb-B2 over-expression was found in many human breast carcinomas. A current approach in treating this type of cancer is inhibition of the activity of this protein (Yamauchi, T. et al., 2000). Similar over-expression of CDK2 has been observed in many cancers, and another approach in cancer treatment is to inhibit the activity of this protein (Buolamwini, J. K., 2000). Another example is PTEN, a tumor suppressor gene, which expresses a phosphatase, mutations in which occur in many different cancers (Li et al, 1997).

Due to the problems in cancer therapy discussed above, it would be useful to be able to therapeutically control protein phosphorylation where needed for normal cell function. It would be useful to develop novel therapeutic methods and anti-tumor agents for controlling cell transformation.

Protein Phosphatases

Protein phosphorylation, a crucial posttranslational modification step controlling many diverse cellular functions, is dependent on the opposing actions of protein kinases and protein phosphatases.

PCT patent application No. WO97/10796 discloses preparing a vector harboring the gene for protein phosphatase 2C_{.} and including regulatory elements to control the expressibility of PP2Cα. This vector is then administered to a patient harboring cancerous (tumor) cells in order to treat the cancer.

It would be advantageous and desirable to provide protein phosphatases to a patient by a non-viral expression system. However, until the invention described in this instant application, there is no delivery system in the art which delivers biologically active polypeptides intracellularly.

Polymers for Drug Targeting

Drug targeting is defined very generally as the concept of delivering an adequate amount of drug to the target site in the body compartment at an appropriate time (Kataoka, 1997).

Several polymer based anticancer agents have now entered the clinic or are currently in clinical trials. The hydroxypropyl methacrylamide (HPMA) copolymer has been studied as a polymeric carrier for low molecular weight anticancer agents (reviewed in Duncan 1992; Duncan et al., 1996). HPMA homopolymer is a hydrophilic, biocompatible polymer originally developed in Czechoslovakia as a plasma expander (Kopecek and Bazilova, 1973).

HPMA copolymers containing doxorubicin (PK1, FCE 28068), doxorubicin and galactosamine (PK2, FCE 28069) and paclitaxel (PNU 166945) are currently in clinical trials (Vasey et al., 1999, Kerr et al., 1998, ten Bokkel Hunink et al., 1998).

Conjugates of polypeptides to certain polymers such as polyethylene glycols have previously been known to enhance penetration of the conjugates into tissues or organs. However, bioactive polypeptides have not previously been bound to HPMA copolymers and used for intracellular delivery of the polypeptide as a therapeutic agent.

Conjugates of HPMA to cell-specific antibody conjugates for targeting of anticancer drug (Flanagan, et al., BBA, 993, 83, 1989; Stastny et al., Eur. J. Cancer 35, 459, 1999) are known. EP 97304070.2 discloses enzyme conjugates and their therapeutic uses with prodrugs, however those applications are for enzymes that exert their therapeutic utility extracellularly.

Nowhere in the background art is it taught or suggested that conjugates of a polypeptide with an acrylamide based copolymer would effect entry of the polypeptide into the cell while retaining the biological activity of the polypeptide.

### SUMMARY OF THE INVENTION

It is an object of the present invention to provide a delivery system capable of delivering a polypeptide or a protein into viable cells while retaining its biological activity. It is a further object of the present invention to provide a complex molecule comprising a conjugate of a polypeptide and a pharmaceutically acceptable polymer, capable of intra-cellular delivery of the biologically active polypeptide. It is a further object of the present invention to provide a complex molecule comprising a conjugate of a polymer capable of being taken up by a cell linked to a biologically active polypeptide, the conjugate capable of achieving intracellular delivery of the polypeptide while retaining the biological activity of said polypeptide.

It is yet a further object of the present invention to provide pharmaceutical compositions comprising these conjugates, and methods of using these conjugates in vivo for therapeutic and diagnostic purposes. The methods of the invention are also suitable for elucidating the activity of unknown polypeptides within cells.

This invention unexpectedly provides medicaments and methods for delivery of biologically active polypeptides, including but not limited to protein phosphatase 2C polypeptides, by means of linking such biologically active polypeptides to polymers, especially HPMA copolymer. An additional unexpected advantage of this invention is the delivery of polypeptides intracellularly (and not just into the interstitium or interstitial space). Furthermore, these polypeptides are delivered to the correct compartment of the cell; in the case of PP2Cα the polypeptide is delivered to the perinuclear region of the cell. Additionally, after intracellular delivery, these polypeptides are surprisingly not immediately degraded intracellularly (e.g., in the lysosomes) but retain biological activity.

The polypeptide of the conjugate will be any polypeptide it is desired to introduce into cells. Generally speaking this means that the invention is particularly useful for polypeptides for which the cells that are the target have no receptors. The polypeptide may be, inter alia, a therapeutic antibody, an intrabody, a toxin, or an enzyme. Most preferred are polypeptides having therapeutic activity, though diagnostic uses are also envisaged.

Nevertheless, it will be appreciated by the skilled artisan that it is possible to use a combination of active targeting (to specific receptors) with passive targeting that is achieved by the conjugates of the present invention, as will be exemplified hereinbelow. The combination of active targeting and passive targeting can involve polymer conjugates carrying more than one polypeptide, or polymer conjugates carrying a polypeptide and another therapeutic agent or targetor.

Encompassed within the scope of the present invention it is possible to use a combination of a polypeptide conjugate administered in conjunction with another therapeutic agent including but not limited to an anti-cancer agent, a therapeutic peptide or a diagnostic reagent. Combination therapies may be administered simultaneously or separately, as the situation warrants or requires.

The polymer can be a homopolymer or a copolymer, including block copolymers, random copolymers and alternating copolymers.

One preferred family of polymers for use in the present invention are N-alkyl acrylamide polymers and include homopolymers and copolymers prepared from monomers of the acrylamide family, such as acrylamide, methacrylamide and hydroxypropylacrylamide. The preferred polymer is a copolymer based on N-(2-hydroxypropyl)-methacrylamide (HPMA), which is prepared by copolymerizing HPMA copolymer with a monomer unit having an oligopeptide side chain (linker) for attachment of the polypeptide, preferably via the NH₂ group of a lysyl and/or arginyl residue.

The preferred HPMA copolymer is a copolymer composed of two repeat units. One is a repeat unit of N-alkyl-acrylamide. The other unit is designed to carry an oligopeptide side chain, which terminates in an end group suitable for attachment to a polypeptide.

Thus, a first aspect of the invention provides a complex molecule comprising copolymer-polypeptide conjugates capable of intracellular delivery of a biologically active polypeptide. One preferred embodiment of the invention provides HPMA copolymer-polypeptide conjugates that achieve intracellular delivery of the polypeptide.

A second aspect of the invention provides complex molecules comprising copolymer-polypeptide conjugates further comprising at least one additional drug or targetor molecule which achieve intracellular delivery of the polypeptide.

A third aspect of the invention provides a pharmaceutical composition comprising a copolymer-polypeptide conjugate capable of intracellular delivery of a biologically active polypeptide. One currently preferred embodiment of the invention provides pharmaceutical compositions of HPMA copolymer-polypeptide conjugates which achieve intracellular delivery of the polypeptide.

Yet another aspect of the invention provides a method for introducing a biologically active polypeptide into a cell, said method comprising the conjugation of the polypeptide to a polymer carrier which achieves intracellular delivery of said polypeptide.

Yet further aspects of the invention provide methods for using the compounds and compositions of the invention for therapeutic and diagnostic purposes in vivo.

The HPMA copolymer—polypeptide conjugate prepared as herein described is used to treat many types of disorders. Particularly preferred uses include anticancer applications, immunosuppressive applications, treatment of genetic disorders or diseases involving enzyme deficiencies, as well as diagnostic uses.

### BRIEF DESCRIPTION OF THE FIGURES

FIG. 1: Diagram of conjugate produced by aminolytic reaction of HPMA copolymer-Gly-Gly-ONp with PP2Cα.

FIG. 2: Typical FPLC trace of free PP2Cα.

FIG. 3: FPLC trace of HPMA copolymer— PP2Cα prepared in phosphate buffer.

FIG. 4: Effect of treatment of HPMA copolymer-PP2C conjugate on B16F10 cells.

FIG. 5: Effect of treatment of HPMA copolymer-PP2C conjugate on M109 cells.

FIG. 6: Effect of treatment by HPMA copolymer-PP2C conjugate on DA3 cells.

FIG. 7: Anti-tumor activity of the HPMA copolymer-PP2C conjugate in vivo.

FIG. 8: Structure of preferred HPMA copolymer. In this Figure, R1 is H or CH₃, R₂ is a lower alkyl or lower hydroxyalkyl group, R₃ is an oligopeptidyl side chain, and m and n are each between 0.1 and 99.9 mole percent, more preferably between 1-99 mole percent, most preferably between 5-95 mole percent.

FIG. 9: The sequence of the DNA encoding novel protein phosphatase 2c, designated protein phosphatase 2C-ζ (zeta). The DNA sequence is SEQ ID NO 2.

FIG. 10: The corresponding amino acid sequence of the DNA sequence of FIG. 9, encoding novel protein phosphatase 2c, designated protein phosphatase 2C-ζ (zeta). The amino acid sequence is SEQ ID NO 3.

FIG. 11. The sequence of the DNA encoding novel human protein phosphatase 2c_{.}β (43 kD). The DNA sequence is SEQ ID NO 4.

FIG. 12. The corresponding amino acid sequence of the DNA sequence of FIG. 11, encoding novel human protein phosphatase 2c_{.}β (43 kD). The amino acid sequence is SEQ ID NO 5.

FIG. 13. Body distribution of ¹²⁵I-labelled free PP2Cα and HPMA-conjugated PP2Cα showed a 3-fold increase in tumor accumulation, and 3-fold longer circulation time of the conjugate.

FIG. 14. Body distribution of ¹²⁵I-labelled free PP2Cα and conjugated PP2Cα showed a significant 4-fold decrease of the conjugate in AUC of liver accumulation.

FIG. 15. Significant decrease in tumor growth rate observed after treatment with HPMA copolymer-PP2C compared to the control group.

FIG. 16. Lack of toxicity of HPMA copolymer-PP2C showing that maximum tolerated dose (MTD) was not attained even at the highest dose used (100 mg/kg).

### DETAILED DESCRIPTION OF THE INVENTION

The present invention relates to a polymer-based intracellular delivery system for protein phosphatases and other polypeptides. This delivery system can be used to deliver polypeptides for anti-tumor, anti-inflammatory, or immunosuppressive therapy, for treatment of genetic disorder or disease, and for therapy of any condition which requires intracellular delivery of polypeptides.

A variety of polymers are suitable for use in the present invention. These include polyvinylpyrrolidone, polyethylene glycol and copolymers thereof, dextrans, methacrylate-vinylpyrrolidone copolymers and others. Polymers suitable for in vivo administration and for conjugation with drugs have been reviewed by Duncan et al., 1992. It will be appreciated that the selected polymer can be synthesized with chemical moieties suitable for attaching the polypeptide. It is to be understood that known conjugates of polypeptides and polymers such as polyethylene glycol-polypeptide conjugates as are known the art are excluded from the present invention.

The polymer can be a homopolymer or a copolymer, including block copolymers, random copolymers and alternating copolymers.

One preferred family of polymers for use in the present invention are N-alkyl acrylamide polymers and include homopolymers and copolymers prepared from monomers of the acrylamide family, such as acrylamide, methacrylamide and hydroxypropylacrylamide. The preferred polymer is a copolymer based on N-(2-hydroxypropyl)-methacrylamide (HPMA), which is prepared by copolymerizing HPMA copolymer with a monomer unit having an oligopeptide side chain (linker) for attachment of the polypeptide, preferably via the NH₂ group of a lysyl and/or arginyl residue.

The HPMA copolymer is reacted with a variety of polypeptides to form a selection of HPMA copolymer-polypeptide conjugates which permit intracellular delivery of the polypeptide.

The preferred HPMA copolymer is a copolymer composed of two repeat units. One is a repeat unit of N-alkyl-acrylamide. The other unit is designed to carry an oligopeptide side chain which terminates in an end group suitable for attachment to a polypeptide.

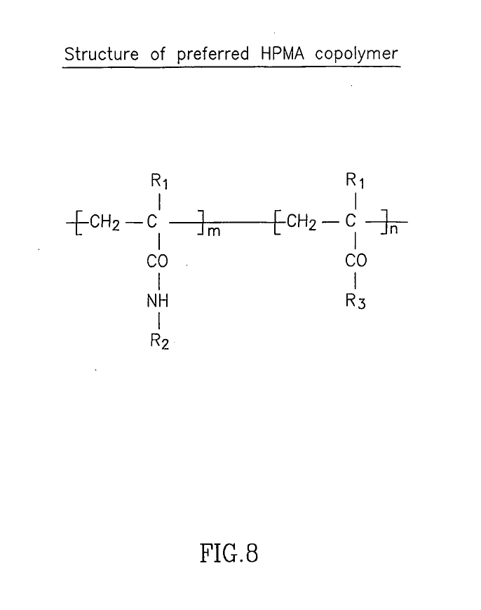

The preferred HPMA copolymer has the general structure shown in FIG. 8.

In FIG. 8, R₁ is H or CH₃, R₂ is a lower alkyl or lower hydroxyalkyl group, R₃ is an oligopeptidyl side chain, and m and n are each between 0.1 and 99.9 mole percent, more preferably between 1-99 mole percent, most preferably between 5-95 mole percent. In the most preferred oligopeptide, R₁ is CH₃ and R₂ is CH₂CHOHCH₃ (hydroxypropyl).

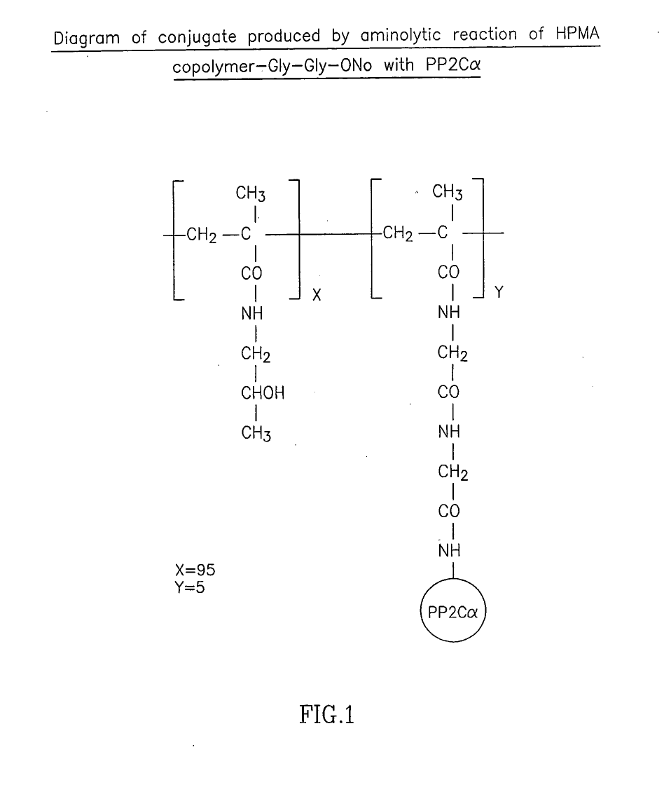

The oligopeptidyl side chain, R₃, is preferably composed of peptidyl or amino acid moieties. Oligopeptide or oligopeptidyl refer to two or more amino acids joined together. Preferred oligopeptides are of the form Gly-(W)ₚ-Gly (SEQ ID NO:1) where p is 0-3 and W is any amino acid. The most preferred oligopeptide of this type is Gly-Gly. This oligopeptidyl side chain is also termed a linker since it links the polypeptide to the HPMA copolymer. An example of a most preferred HPMA copolymer bound to a protein phosphatase 2c polypeptide is shown in FIG. 1. The Gly-Gly linker is bound directly to the PP2C via the NH₂ group of a lysyl and/or arginyl residue of the PP2C by a non-specific aminolytic reaction.

The term “protein phosphatase” includes all of the enzymes in the protein phosphatase super-family of enzymes, including tyrosine phophatases and serine/threonine phophatases.

The term “protein phosphatase 2C” includes all of the protein phosphatase 2C (PP2C; also termed PPM1) family of enzymes. Known PP2C isoenzymes (isoforms) are PP2Cα, PP2Cβ, PP2Cγ (also called FIN 13), PP2Cδ, Wip1, Ca⁺⁺-calmodulin dependent kinase II phosphatase and NER PP-2C. A novel PP2C isoenzyme, designated PP2C-zeta, is disclosed in this application. Also disclosed for the first time is human PP2Cβ_{.} (43 kDaltons), which has been cloned and the sequence determined. There are many forms of _{.} PP2Cβ (43 kD-53 kD) due to alternative splicing. It is envisaged that other isoenzymes may be found and they are also included in the term protein phosphatase 2C.

Compositions of the invention comprise a carrier and a pharmaceutically effective amount of a polymer capable of being taken up by a cell, said polymer linked to a polypeptide. In a preferred embodiment the linkage is by means of a linker. In a more preferred embodiment the linker is not degraded under physiological conditions. In a most preferred embodiment the non-degradable linker comprises a peptide, more preferably a dipeptide, most preferably Gly-Gly.

In a preferred embodiment the polymer is an N-alkyl acrylamide polymer.

The N-alkyl acrylamide polymer may be a homopolymer or preferably a co-polymer, most preferably the co-polymer derived from HPMA copolymer.

In a most preferred embodiment of the invention, the polypeptide linked to the polymer in the composition is glucocerebrosidase. Use of the composition in treatment of Gaucher's disease is envisaged.

In other preferred embodiments the polypeptide linked to the polymer in the composition of the invention is an anti-tumor polypeptide, an anti-inflammatory polypeptide, a polypeptide for treatment of genetic disease, a polypeptide for therapy of auto-immune disease, a polypeptide for production of early abortion, a polypeptide for anti-reocclusion or to prevent re-stenosis or a polypeptide for immunosuppressive therapy, preferably for use in a transplantation procedure, most preferably for corneal transplantation.

Medical devices coated with the compositions of this invention are also envisaged, most preferably a stent coated with a polypeptide for anti-reocclusion or to prevent re-stenosis.

Compositions of this invention may additionally comprise a protein localization signal, preferably an internal protein localization signal.

Embodiments include methods of treating a subject suffering from a disorder, which comprise administering to the subject an amount of the composition of the invention effective to treat the disorder.

Embodiments include methods of treating a symptom in a subject which comprises administering to the subject an amount of the composition of the invention effective to treat the symptom.

Embodiments include methods of contacting a eukaryotic cell with a composition comprising a carrier and a polymer capable of being taken up by a cell linked to a polypeptide, where the polypeptide enters the cell and performs enzymatic activity. In a preferred embodiment, the enzymatic activity is glucocerebrosidase.

As used herein, the term “carrier” encompasses any of the standard pharmaceutical carriers. Such carriers are well known in the art and may include, but are in no way and are not intended to be limited to, any of the standard pharmaceutical carriers such as phosphate buffered saline solutions, water, emulsions such as oil/water emulsion, suspensions, and various types of wetting agents. Typically, such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives, preservatives and the like, or other ingredients.

Compositions (medicaments) comprising such carriers are formulated by well-known conventional methods. The compositions of this invention may include sterile solutions, tablets, coated tablets, capsules, pills, ointments, creams, lotions, gels, suppositories, drops, liquids, sprays and powders or any other means known in the art.

As regards dosage, the medicament should be administered in an amount of 0.1 to 2000 mg of polypeptide equivalent/Kg body weight per day, preferably 1.0 to 1000 mg/Kg body weight per day, most preferably 1.0 to 100 mg/Kg body weight per day.

The administration of the compositions of this invention may be effected by any of the well-known methods, including, but not limited to, intravenous, intramuscular, intravesical, intraperitoneal, topical, subcutaneous, rectal, vaginal, ophthalmical, pulmonary, nasal, oral and buccal administration, by inhalation or insufflation (via the nose or mouth) or by administration as a coating to a medical device (e.g. a stent).

The biologically active polypeptides of the subject invention may be constructed using recombinant technology. One means for obtaining the polypeptides is to express nucleic acid encoding the polypeptide in a suitable host, such as bacterial, yeast or mammalian cell, using methods well known in the art, and recovering the polypeptide after it has been expressed in the host. The nucleic acid expressed may be genomic DNA, cDNA, synthetic DNA, inter alia. In addition, non-recombinant techniques such as chemical synthesis may be used to obtain biologically active polypeptides of the subject invention.

As used herein, the term “polypeptide” refers to a chain of amino acids joined together, preferably 30 or more amino acids, more preferably 50 or more amino acids, most preferably 100 or more amino acids. The amino acids are preferably chemically joined by peptidyl bonds. However, the term “polypeptide” also includes peptidomimetics, such as polypeptoids and semi-polypeptoids which are peptide analogs, which may have, for example, modifications rendering the polypeptides more stable under physiological conditions. Such modifications include, but are not limited to, cyclization, N-terminus modification, C-terminus modification, peptide bond modification, including, but not limited to, one or more of the following modifications: CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art, and are specified, for example, in Quantitative Drug Design, C. A. Ramsen Ed., Chapter 17.2, F. Choplin Pergamon Press (1992), which is incorporated by reference as if fully set forth herein.

The polypeptides of the subject invention also include homologs of the polypeptides. Such homologs have substantially the same amino acid sequence and biological activity as the polypeptide itself. Examples of homologs are deletion homologs containing less than all the residues of the polypeptide, substitution homologs wherein one or more amino acid residues are replaced by other residues, and addition homologs wherein one or more amino acid residues are added to the polypeptide. Substantially the same amino acid sequence is herein defined as the addition, deletion or substitution of up to 20% of the amino acid in the polypeptide. All such homologs share the biological activity of the polypeptide of the invention. Additions or deletions of amino acids may occur at the N-terminus of the polypeptide, at the C-terminus of the polypeptide or within the sequence. Substitutions may occur anywhere in the sequence, and substitutions which do not affect the biological activity are known to those skilled in the art. Substitutions preferably encompass up to 10 amino acid residues in accordance with the homologous or equivalent groups described by e.g. Lehninger, Biochemistry, 2^{nd} edition Worth Pubs (1975); Creighton, Protein Structure, a practical Approach, IRL press at Oxford Univ. Press, Oxford, England(1989); and Dayhoff, Atlas of Protein Sequence and Structure 1972, National Biomedical Research Foundation, Maryland (1972).

The term “tumor” as used herein encompasses all types of tumors, preferably solid and semi-solid tumors, including inter alia, melanoma, carcinoma, lymphoma, and blastoma. The term “tumor” encompasses primary tumors, secondary tumors, and metastases thereof in the same organ or in another organ.

The term “treatment of a tumor” or “anti-tumor” as used herein refers to a treatment or a composition which retards the proliferation of a tumor and/or causes regression of a tumor.

The HPMA copolymer used in the experimental work described in the Examples was obtained from Polymer Labs, U.K. HPMA may also be made by methods known in the art, for example, as described in U.S. Pat. No. 5,965,118 (Duncan, Ruth et al.) and Duncan et al., 1987 inter alia.

Most therapeutic regimes in modern chemotherapy involve the simultaneous administration of a number of anti-neoplastic agents. For example, the clinical utility of doxorubicin is predominantly in combination chemotherapy (Bonadonna et al., 1974); when used in combination it often synergizes, yielding longer remissions than are observed when it is used as a single agent.

Thus it is envisaged that the anti-tumor medicaments of the subject invention may be used in conjunction with other anti-tumor agents.

### EXAMPLES

The Examples which follow are set forth to aid in understanding the invention but are not intended to, and should not be construed to, limit its scope on any way.

### Example 1

### Cloning and Purification of Human PP2Cα_{.}

Human PP2Cα_{.} cDNA isolated from human testis marathon-ready cDNA library (Clontech), and rat PP2Cα_{. .} cDNA isolated from rat embryo cDNA library (PCT patent application No. WO97/10796), were each separately cloned into the E. coli expression vector pET28b (Novagen). The His-tag sequence was removed from the vector by restriction, yielding expression of the authentic human (or rat, respectively) PP2Cα_{. .} sequence with the typical Mg²⁺ dependent phosphatase activity. A BL21/DE3 Strain of E. coli was separately transformed by each recombinant plasmid (encoding human or rat PP2Cα_{. .}, respectively) and produced high levels of soluble recombinant human PP2C_{.} and rat PP2C_{.}, respectively, as observed by SDS-PAGE. The transformants were grown in 500 ml LB medium supplemented with 50 μg/ml kanamycin. When growth reached an optical density of 0.6 at 600 nm, 0.1 mM of isopropyl-1-thio-b-D-galactopyranoside was added and the cultures were grown overnight at 30° C. Cells were harvested by centrifugation of 10 min at 6000 g, washed once in 20 mM Tris-HCl, 150 mM NaCl pH 7.5 and resuspended in buffer A (20 mM Tris-HCl, 20 mM NaCl, 1 mM EGTA pH 7.5) supplemented with protease inhibitor cocktail (Boehringer Mannheim). Cells were lysed by sonication (Heat Systems) and cell debris was pelleted by centrifugation 30 min at 25000 g. The supernatant, containing PP2C was diluted with an equal volume of buffer A, filtered on a 0.45 m filter and applied to a 1.5×17 DEAE-Sepharose Fast Flow anion exchange column (Amersham Pharmacia Biotech) equilibrated in buffer A. PP2C_{.} was eluted by a 100-500 mM NaCl gradient of 150 ml. Fractions containing PP2C_{.} were pooled, adjusted to 1.5 M NaCl and applied to a 1.5×17 Phenyl-Sepharose 6 Fast Flow (high sub) hydrophobic column (Amersham Pharmacia Biotech) equilibrated in 20 mM Tris-HCl, 1.5 M NaCl, 1 mM EGTA. PP2C_{.} was eluted with Buffer A, concentrated on Vivaspin 6 concentrator 10000 MWCO (Sartorius) to 20 mg/ml and chromatographed on 16×60 Superdex 200 size exclusion column (Amersham Pharmacia Biotech). Fractions of highest purity were pooled and used for conjugation.

The recombinant polypeptide PP2C_{.} may also be obtained as described in PCT patent application No. WO97/10796.

### Example 2

### Preparation of the HPMA Copolymer-PP2C Conjugate

A. Optimization Method of Polymer-Enzyme Conjugation

HPMA was obtained from Polymer Labs, U.K.

HPMA copolymer-Gly-Gly-ONp was conjugated to PP2Cα_{. .} via an aminolytic reaction, yielding the conjugate depicted in FIG. 1. This conjugate comprises HPMA copolymer bound to a terminal NH₂ group of a lysyl and/or arginyl residue in PP2C_{.} via a Gly-Gly linker. A variety of methods was used to optimize the polymer-enzyme conjugation. These methods are summarized in Table 1.

TABLE 1
Variables examined in synthesis of HPMA copolymer-PP2C.
conjugate.

| | HPMA | | | | |
|---|---|---|---|---|---|
| | copolymer- | | Total | | Conjugate |
| | Gly-Gly- | Raising | reaction | | Activity |
| | ONp: PP2C. | pH Time | Time | | (pmol/min/ |
| Batch | Ratio | (h) | (h) | Solvent | mg) |
| RSF-1 | 1:1 | | 30 min | HPMA/DMSO | 18,032 |
| | | | | PP2C./DMSO | |
| RSF-2 | 1:1 | | 1 | HPMA/DMSO | 17,688 |
| | | | | PP2C./DMSO | |
| RSF-3 | 1:1 | | 6 | HPMA/DMSO | 17,158 |
| | | | | PP2C./DMSO | |
| RSF-4 | 1:2 | | 6 | HPMA/DMSO | 14,396 |
| | | | | PP2C./DMSO | |
| RSF-5 | 1:1 | 30 min | 30 min | HPMA/DDH₂O | 266,000 |
| | | (immediate | | PP2C./PBS | |
| | | increase | | | |
| | | to pH | | | |
| | | 8.5) | | | |
| RSF-6 | 1:1 | 2 h | 24 | HPMA/DDH₂O | 6,411,333 |
| | | (pH | | PP2C./PBS | |
| | | raised | | | |
| | | to | | | |
| | | 8.5) | | | |
| RSF-7 | 1:1 | 1 (pH | 24 | HPMA/DDH₂O | 692,409 |
| | | raised | | PP2C./PBS | |
| | | to | | | |
| | | 8.5) | | | |
| RSF-8 | 2:1 | 1 (pH | 24 | HPMA/DDH₂O | 1,043,088 |
| | | raised | | PP2C./PBS | |
| | | to | | | |
| | | 8.5) | | | |
| RSF-9 | 1:1 | | 24 | HPMA/DMSO | 998,456 |
| | | | | PP2C./DMSO | |
| RSF-10 | 1:2 | — | 24 | HPMA/DMSO | 1,119,600 |
| | | | | PP2C./DMSO | |
| RSF-11 | 1:1 | — | 25 | HPMA/DMSO | 2,299 |
| | | | | PP2C./DMSO | |
| RSF- | 1:1 | — | 24 | HPMA/DDW | 3,392,767 |
| 15 (1) | | | | PP2C./Am- | |
| | | | | monium | |
| | | | | Bicarbonate | |
| | | | | (AB) | |
| RSF- | 1:1 | — | 24 | HPMA/DMSO | 1,490,517 |
| 15 (2) | | | | PP2C.//DMSO | |
| RSF- | 1:1 | — | 24 | HPMA/AB | 2,147 |
| 15 (3) | | | | PP2C./AB | |
| RSF-16 | 1:1 | — | 3 | HPMA/DMSO | 127,431 |
| | | | | PP2C./dry | |
| | | | | PBS + DMSO | |
| RSF-h20 | 1:1 | 2 h | 24 | HPMA/DDW | 1,955,093 |
| (human) | | (pH | | PP2C./PBS, | |
| | | raised | | MgCl₂, EGTA | |
| | | to | | | |
| | | 8.5) | | | |
| RSF-h21 | 1:1 | 2 h | 24 | HPMA/DDW | 11,388 |
| (human) | | (pH | | PP2C./PBS, | |
| | | raised | | MgCl₂, EGTA | |
| | | to | | | |
| | | 8.5) | | | |
| RSF-22 | 1:1 | 2 h | 24 | HPMA/DDW | 4,942,152 |
| | | (pH | | PP2C./PBS, | |
| | | raised | | MgCl₂, EGTA | |
| | | to | | | |
| | | 8.5) | | | |
| RSF-23 | 1:1 | 2 h | 24 | HPMA/DDW | 1,002 |
| mutant | | (pH | | PP2C./PBS, | |
| | | raised | | MgCl₂, EGTA | |
| | | to | | | |
| | | 8.5) | | | |
| RSF-25 | 1:1 | 2 h | 24 | HPMA/DDW | 7,774 |
| mutant | | (pH | | PP2C./PBS, | |
| | | raised | | MgCl₂, EGTA | |
| | | to | | | |
| | | 8.5) | | | |
| RSF-h26 | 1:1 | 2 h | 24 | HPMA/DDW | 10,850,424 |
| (human) | | (pH | | PP2C./PBS, | |
| | | raised | | MgCl₂, EGTA | |
| | | to | | | |
| | | 8.5) | | | |
| RSF-27v | 1:1 | | 1 | HPMA/DMSO | 20,811,111 |
| | | | | PP2C.//DMSO | |
| RSF-27d | 1:1 | | 1 | HPMA/DMSO | 4,780,473 |
| | | | | PP2C.//DMSO | |
| RSF- | 1:1 | | 1 | HPMA/DMSO | 2,164,093 |
| h28v | | | | PP2C.//DMSO | |
| (human) | | | | | |
| RSF- | 1:1 | | 1 | HPMA/DMSO | 1,309,864 |
| h28d | | | | PP2C.//DMSO | |
| (human) | | | | | |
| RSF-29d | 1:1 | 2 h | 24 | HPMA/DDW | 8,470,740 |
| | | (pH | | PP2C./PBS, | |
| | | raised | | MgCl₂, EGTA | |
| | | to | | | |
| | | 8.5) | | | |
| RSF- | 1:1 | 2 h | 24 | HPMA/DDW | 7,909,390 |
| h30d | | (pH | | PP2C./PBS, | |
| (human) | | raised | | MgCl₂, EGTA | |
| | | to | | | |
| | | 8.5) | | | |

Abbreviations used in the above Table are as follows: PBS = Phosphate buffer solution, pH 7.4, 0.05 M; DDW = double deionized water; DMSO = dimethylsulfoxide.

In general, the methods used to form the conjugate were as follows: All mixtures were prepared in the dark, at 4° C. while stirring. All batches were followed by UV spectrophotometer analysis and all showed p-nitrophenol release (shift of peak from 270 nm to 400 nm). All reactions were terminated by addition of 1-amino-2-propanol. The formation of the conjugate was analyzed by SDS PAGE analysis and FPLC.

The following two methods are described in detail below:

(A). Conjugation in Phosphate Buffer

HPMA copolymer-Gly-Gly-ONp was dissolved in double deionized water (DDW) (2 mg/ml) and the solution of PP2C_{.} in 0.05 M phosphate buffer, pH 7.4 (2 mg/ml) was added dropwise at 4° C. under stirring. The reaction mixture was stirred in the dark for 30 min. Then the pH was carefully raised during a 4 h period by adding saturated tetraborate buffer up to pH 8.5. The mixture was stirred for another 4 h and the reaction was completed by adding 1-amino-2-propanol (half of the equivalent amount in relation to the original ONp groups) in order to remove unreacted ONp groups. The final yellow solution was transferred to a VivaSpin (10 KDa MW cut-off) column in order to remove any low MW compounds present in the solution (free ONp, 1-amino-2-propanol, tetraborate salts). The VivaSpin was centrifuged at 4° C. at 3000 g for 30 min. This procedure was repeated, while adding phosphate buffer at each time, until no ONp groups were visible (no yellow color left). The mixture was concentrated to a final volume of 500 μl. Another method of purification was dialysis of some of the batches mentioned above against DDH₂O or PBS in a ‘Snake Skin’ dialysis membrane 10 kD MW cut-off.

(B) Conjugation in DMSO

PP2Cα_{. .} in PBS solution was lyophilized and redissolved in DMSO. HPMA-Gly-Gly-ONp was dissolved in dimethylsulfoxide (DMSO) (2 mg/ml) and the solution of PP2Cα_{. .} in DMSO (2 mg/ml) was added dropwise under stirring. The reaction mixture was stirred in the dark for 10 min. The mixture was stirred for another 20 min and the reaction was completed by adding 1-amino-2-propanol (½ the equivalent amount in relation to the original ONp groups) in order to remove unreacted ONp groups. The final yellow solution was transferred to a VivaSpin (10 kD MW cut-off) column in order to remove any low MW compounds present in the solution. The VivaSpin was centrifuged at 4° C. at 3000 g for 30 min. This procedure was repeated, while adding phosphate buffer at each time, until no ONp groups were visible (no yellow color left). The mixture was concentrated to a final volume of 500 ml. Another method of purification was dialysis of some of the batches mentioned above against DDH₂O or PBS in a ‘Snake Skin_{.} dialysis membrane 10 kD MW cut-off.

II. Analysis of the HPMA Copolymer-PP2C Conjugate:

A. Free PP2Cα_{. .}

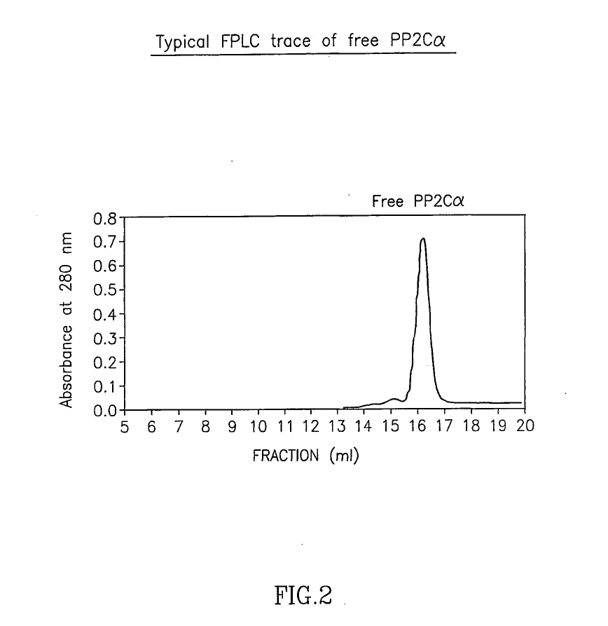

Free PP2Cα_{.} was run through an FPLC column and showed a peak in fractions 16 and 17 using a UV detector at 280 nm. The results are shown in FIG. 2.

B. The HPMA Copolymer-PP2C Conjugate

The HPMA copolymer-PP2C conjugate was analyzed by FPLC. 200 ml solution (recovered from the VivaSpin in Section I above) was passed through the FPLC column (Superdex 200 HR 10/30 from Amersham Pharmacia Biotec) under the following conditions:

The buffer was 0.01 M phosphate buffer with 0.15 M NaCl, pH 7.4, the flow rate was 0.5 ml/min, the detector was UV-M, 280 nm, 0.5 AUFS, and the software was FPLC director® version 1.10. (These are the same conditions as were used for analysis of the free PP2C_{.} above.) Fractions (1.0 ml) were collected and tested for activity.

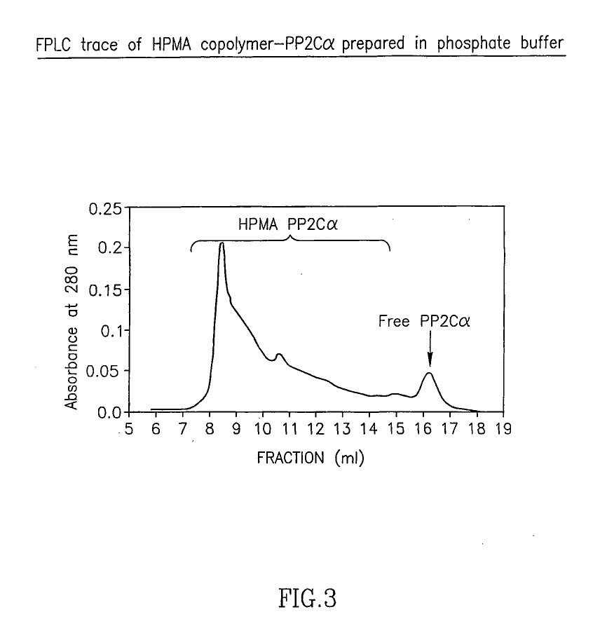

FIG. 3 shows the results of analysis of the HPMA copolymer-PP2C conjugate. The yield of this FPLC step (was calculated from determination of the area under the curve (AUC) and determination of amount of protein (by bicinchoninic acid (BCA) assay). The yield was found to be 55%.

III. Determination of Phosphatase Activity

Phosphatase activity of free and of conjugated PP2C was determined by the Malachite-Green assay by the method of Marley et al., 1998 and Baykov et al., 1988. Using the phosphopeptide FLRTpSCG as a substrate, the amount of free phosphate generated by dephosphorylation is determined by measuring the absorbance of a molybdate:malachite-green:phosphate complex which is proportional to the free phosphate concentration. The assay is carried out in 96-well microtiter plates (1/2 area, flat bottom) in a volume of 30 ml, at 30° C. for 25 min. The reaction mixture contains 0.4 mM substrate in 50 mM Tris-HCl, 0.1 mM EGTA, 30 mM MgCl₂ pH 7.5 and 5-20 ng PP2C. Following incubation, the reaction volume is brought to 100 μl and 25 μl of the ammonium molybdate:malachite green mixture is added. Absorbance at 630 nm is compared to a standard curve constructed with known amounts of free phosphate. Phosphatase activity is expressed as the amount of phosphate released per min per mg PP2C. The results are summarized in Table 1 above.

Combination of Active and Passive Targeting.

In order to increase efficiency and rate of HPMA-PP2C transduction into tumor cells the following constructs are also suitable:

1. PP2C-GnRH Fusion Protein

GnRH-like receptor was found to be overexpressed in adenocarcinoma and has been suggested as a diagnostic marker that distinguishes between adeno and non-adenocarcinomas.

To utilize this receptor as a means for receptor mediated endocytosis, we constructed a fusion protein consisting of GnRH-PP2C. The construct which was His-tagged was purified on a Ni-Agarose column and conjugated to HPMA copolymer-Gly-Gly-ONp (RSF53). Alternatively, GnRH peptide and PP2C were both conjugated in parallel to HPMA copolymer (RSF58). The conjugates were analyzed by FPLC and tested for phosphatase activity. Cultured 293 cells (bearing the GnRH receptor) were treated with increasing doses of HPMA-GnRH-PP2C.

2. Poly-Arg-PP2C. Recently, arginine-rich peptides were suggested in the literature as a means of transduction of proteins into the cells. We have built a poly-Arg-PP2C construct in order to facilitate the introduction of HPMA-poly-Arg-PP2C into the cells by passive and active means. We are currently working on the purification protocol of this construct, conjugation to the HPMA copolymer via aminolysis and characterization. Preliminary results of XTT assay of cells treated with this conjugate shows the advantage of the combination of passive pinocytosis targeting with the active properties of the poly-Arg.

3. Poly-Lys-PP2C. fusion protein was constructed in our laboratory in order to improve the conjugation to HPMA copolymer and avoid steric hindrance and reduction of activity.

4. Fluorescent labeling or other suitable tagging of conjugates are constructed in order to facilitate quantitation and distribution studies. For example FITC-labeling of PP2C may be used to quantitate amounts of conjugate delivered into the cells.

### Example 3

### Effect of HPMA Copolymer-PP2C Conjugate on Plating Efficiency of Melanoma Cells

The effect of HPMA copolymer-PP2C conjugate, prepared as described in Example 2, on the plating efficiency of melanoma cells was studied as follows:

Plating Efficiency Assay

B16F10.9 melanoma cells (100) were seeded on a 24 well plate containing DMEM medium+10% fetal calf serum (FCS)+Penicillin/Streptomycin antibiotics. HPMA-PP2Cα conjugate (100-200 μg PP2Cα-polypeptide equivalent) was added to some of the wells in the 24 well plate. These wells were compared to untreated cells in parallel wells. Plates were left in an incubator for 8 days in order to test the ability of the melanoma cells to form colonies in the presence and absence of the HPMA copolymer-PP2C conjugate. All cells were fixed to the plate with 100% methanol for 20 min. Following fixation cells were washed with running water and the wells were filled with 10% aqueous Giemsa solution that had been filtered through Whatman paper (1 mm). The dye was left at room temperature for 20 min. Plates were then washed with running water, dried and colonies were counted. The calculations were made on the basis of average number of colonies developed.

TABLE 2
Colony count of melanoma cells in the presence and absence
of the HPMA copolymer-PP2C conjugate

HPMA copolymer-PP2C. conjugate (μg/ml)

| | 200 | 100 |
|---|---|---|
| 2 days | 0 | 11 |
| 8 days | 0 | 7 |
| Untreated | 54 | 49 |

Medium including the conjugate was replaced with fresh medium without conjugate after 2 days and after 8 days, indicative of stability of the conjugate.

These results showed that the HPMA copolymer-PP2C conjugate inhibits the capability of tumor cells to replicate and form colonies. These results also showed that 2 days of treatment has similar effect to 8 days of treatment. Subsequent experiments showed that incubation of the HPMA copolymer-PP2C conjugate for as little as a six-hour period gave similar results.

### Example 4

### Effect of the HPMA Copolymer-PP2C Conjugate on Various Tumor Cell Lines

The effect of the HPMA copolymer-PP2C conjugate on the proliferation of tumor cell lines was studied. These cell lines were B16F10.9, M109 and DA3 cells. The B16F10.9 cells are melanoma cells, the M109 are colon carcinoma cells, and the DA3 cells are mammary carcinoma cells.

The effect of the HPMA copolymer-PP2C conjugate, prepared as described in Example 2, was studied as follows:

Cell Proliferation Assay With XTT Reagent.

The use of tetrazolium salts, such as MTT, commenced in the 1950s and is based on the fact that live cells reduce tetrazolium salts into colored formazan compounds. The biochemical procedure is based on the activity of mitochondrial enzymes which are inactivated shortly after cell death. This method was found to be very efficient in assessing the viability of cells. A calorimetric method based on the tetrazolium salt, XTT, was first described by P. A. Scudiero (Scudiero, 1988). Herein, a commercial kit purchased from Biological Industries Co., Israel (Beit Haemek(1990)Ltd.) was used. Whilst the use of MTT produced a non-soluble formazan compound which necessitated dissolving the dye in order to measure it, the use of XTT produces a soluble dye.

Assay Procedure:

B16F10.9, M109 and DA3 cells (50-800) were cultivated in a flat bottom 96-well plate. To each well 100 μl of growth media was added. The cells were incubated in a CO₂ incubator at 37° C., and were used for the proliferation assay after 24 h. At this point, cells were treated with HPMA copolymer-PP2Cα_{. .} conjugate and left in the incubator for 72 h.

XTT reagent solution and the activation solution were defrosted immediately prior to use in a 37° C. bath. Reagents were swirled gently until clear solutions were obtained. Activation solution (100 μl) was added to 5 ml XTT reagent. 50 μl of the reaction solution were added to each well. Plates were incubated for 3 h, shaken gently to evenly distribute the dye in the wells. Absorbance was measured with a spectrophotometer (ELISA reader) at a wavelength of 450-500 nm. In order to measure the specific effect of the conjugate, a reference absorbance wavelength of 630-690 nm was used (to measure non-specific readings).

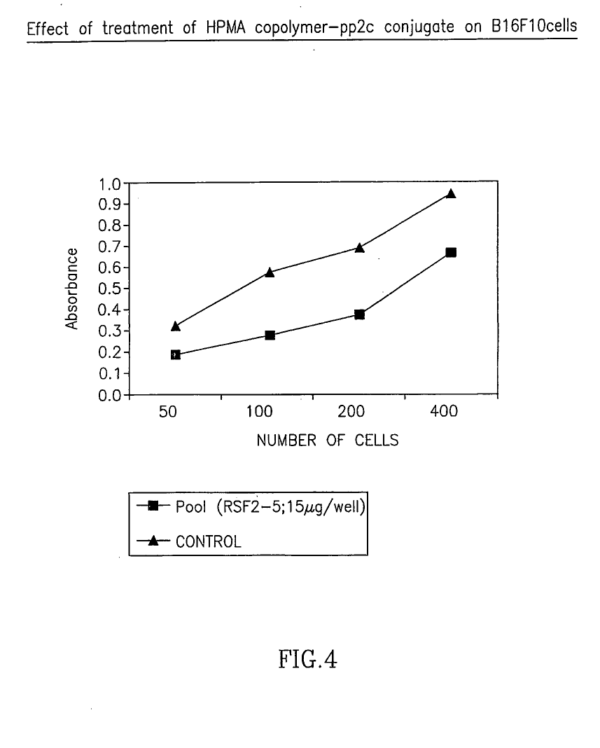

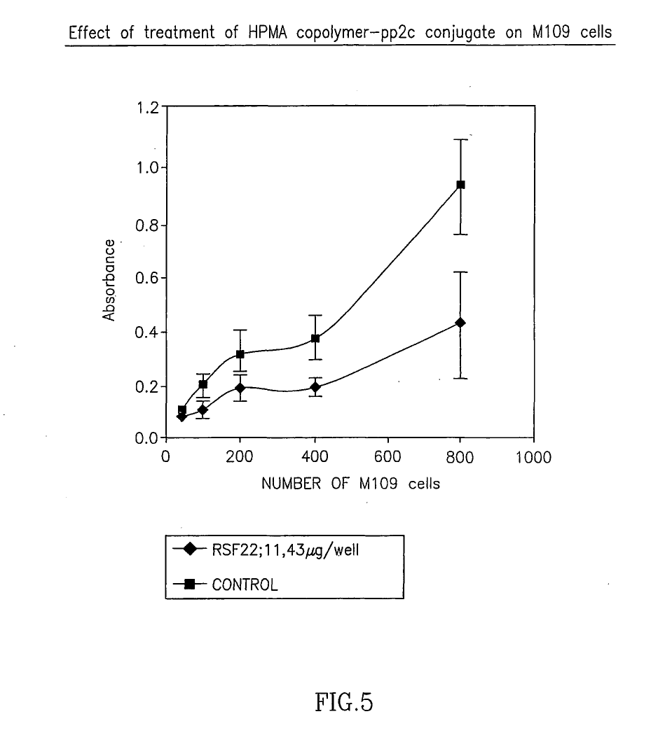

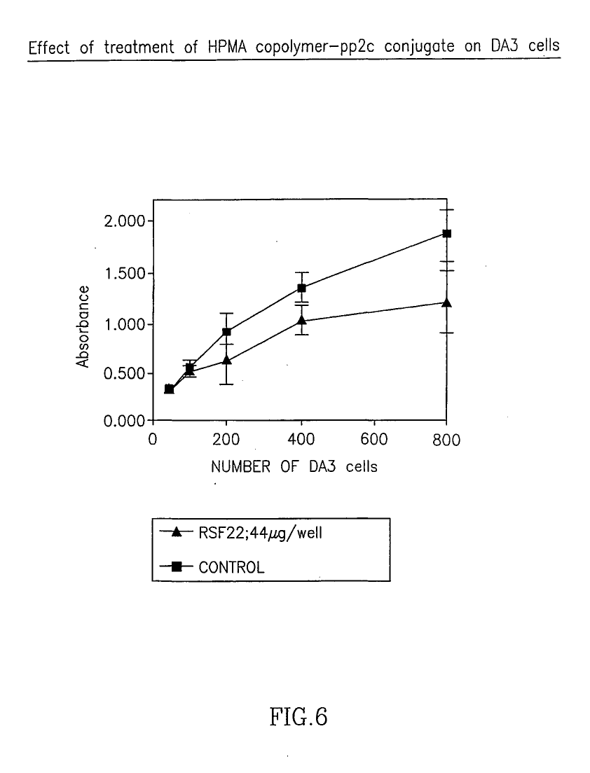

FIG. 4 shows effect of treatment on B16F10 cells. FIG. 5 shows effect of treatment on M109 cells. FIG. 6 shows effect of treatment on DA3 cells.

In all three cases, treatment with the HPMA copolymer-PP2C conjugate reduced cell proliferation compared to the control samples, i.e., HPMA copolymer-PP2C conjugate has an anti-proliferative effect on tumor cells.

### Example 5

### Intracellular Localization of the HPMA Copolymer-PP2C Conjugate

The localization of the HPMA copolymer-PP2C conjugate, prepared as described in Example 2, was studied as follows:

Immunofluorescence Assay

B16F10 cells were applied on slides, then treated with HPMA-PP2Cα_{.} conjugate and incubated for 2 h. The cells were then stained with a specific monoclonal antibody for PP2Cα_{.} (recognizing PP2Cα_{.} and not PP2Cβ_{.}) which is conjugated to fluorescein isothiocyanate (FITC). The antibody was obtained as described in PCT patent application No. WO97/10796.

The results are tabulated in Table 3.

TABLE 3
Immunofluorescence of intracellular passage of HPMA PP2Cα.
to B16F10 murine melanoma cells

| | Green | Total No. | % Green |
|---|---|---|---|
| Group | Cells | Cells | Cells |
| Untreated | 0 | 56 | 0 |
| HPMA-PP2Cα. 6(aq.) 200 μg | 29 | 49 | 59.2 |
| HPMA-PP2Cα. 11(aq.) 200 μg | 31 | 34 | 91.2 |
| HPMA-PP2Cα. . 6(aq.) 400 μg | 65 | 68 | 95.6 |
| HPMA-PP2Cα. .11(DMSO) 400 μg | 70 | 87 | 80.5 |
| HPMA-PP2Cα. . 6(DMSO) 400 μg | 49 | 50 | 98 |

These results showed that the HPMA copolymer-PP2C conjugate entered most of the cells tested.

Confocal microscopy analysis revealed that the dye was efficiently internalized within two hours, in the perinuclear region. The localization of the HPMA copolymer-PP2C conjugate within the cell (in the perinuclear region) was similar to the localization of naturally occurring (endogenous) PP2C.

This most unexpected result showed that the HPMA copolymer-PP2C conjugate can enter the cell and deliver the PP2C to the correct region of the cell.

Similar experiments to those shown in Table 3 were performed using a fusion protein comprising GFP (Green Fluorescence Protein)—PP2C conjugated to HPMA. (The fusion protein comprising GFP—PP2C was expressed from a fusion gene construct.) It was found that more than 90% of the cells became green, indicating that the fusion protein was introduced to almost all the cells.

### Example 6

### Evaluation of Antitumor Activity of the HPMA Copolymer-PP2C Conjugate

Male C57BL/6J mice were inoculated with 10⁵ viable B16F10 melanoma cells subcutaneously. The tumor was allowed to establish until the area was approximately 20-50 mm² as measured by the product of two orthogonal diameters.

Animals were injected intravenously via the tail vein in a single treatment with HPMA-PP2C_{.} conjugate. The PP2C_{.} batches used were RSF-h26 and RSF-29d at an equivalent dose of 20 mg/Kg polypeptide equivalent in saline, prepared as described in Example 2. Additional groups of animals were treated with saline (100 μl intravenously) as control. Each group consisted of 6 mice.

Animals were weighed and the tumor measured daily. Animals were monitored for general health, weight loss and tumor progression. There was no weight loss, indicating that dose escalation and/or repeated dosage is possible.

Mice were culled when the tumor reached or surpassed the size of 300 mm^{2.} At termination the animals were examined by post-mortem and the tumors were dissected and weighed.

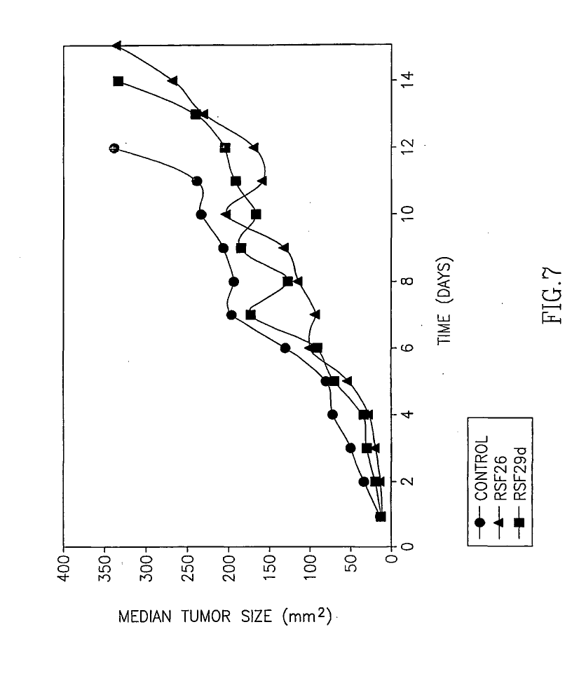

The results are summarized in FIG. 7. FIG. 7 shows that growth of the tumor was much slower in the mice treated with the conjugate. Note that in this experiment the conjugate was administered once only on day zero. It is anticipated that repeat treatments with the conjugate can cause complete regression of the tumor, without fear of immunogenicity.

### Example 7

### Novel Protein Phosphatase 2C, Designated Protein Phosphatase 2Cζ (Zeta)

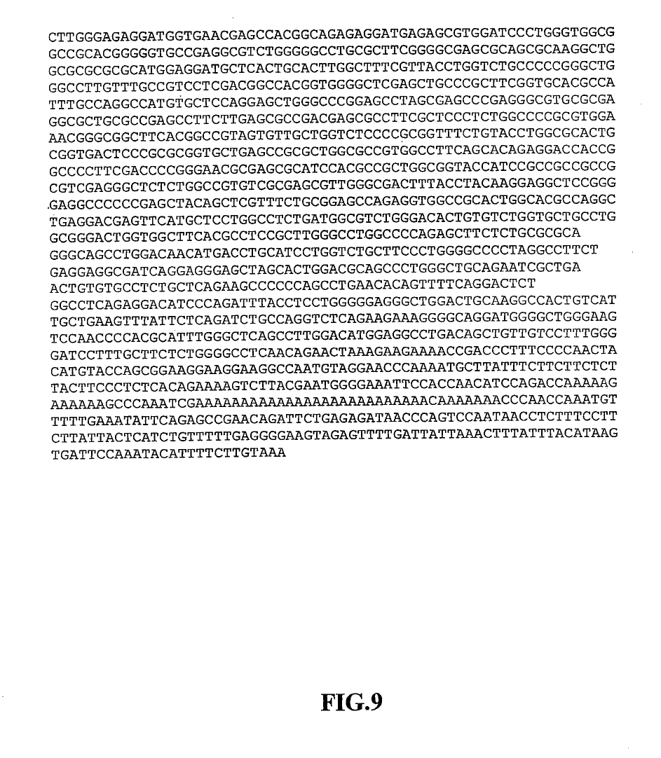

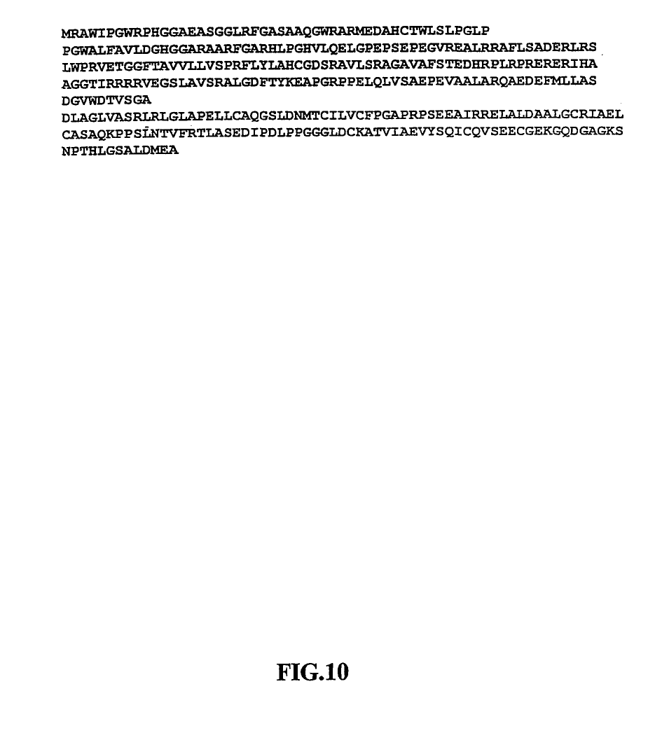

A novel protein phosphatase 2C, designated protein phosphatase 2Cζ (protein phosphatase 2C-zeta), was found. It was cloned from human cells, and sequenced. The sequence of the DNA is recited in FIG. 9, and the corresponding amino acid sequence is recited in FIG. 10. The DNA sequence is SEQ ID NO 2, and the corresponding amino acid sequence is SEQ ID NO 3.

### Example 8

### Novel Human Protein Phosphatase 2C (43 kD)

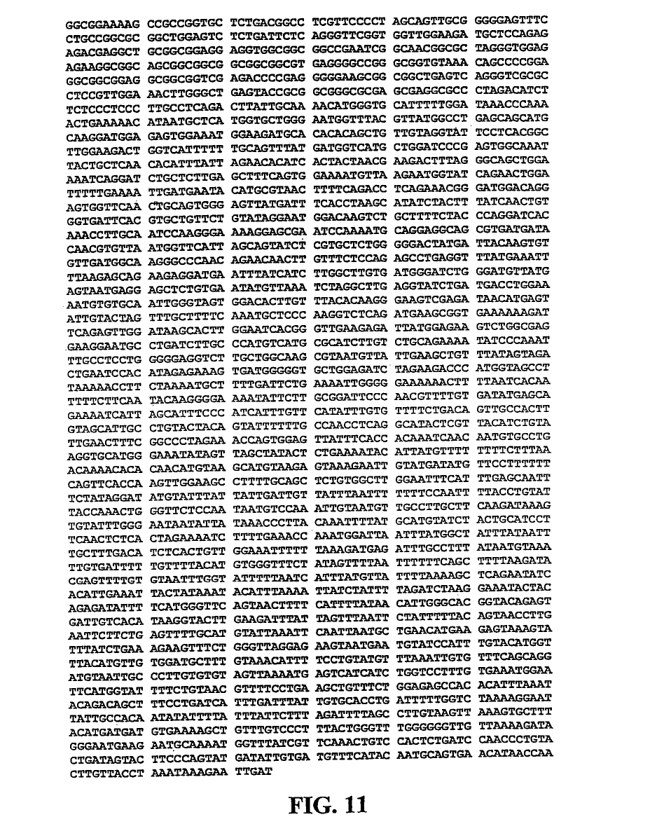

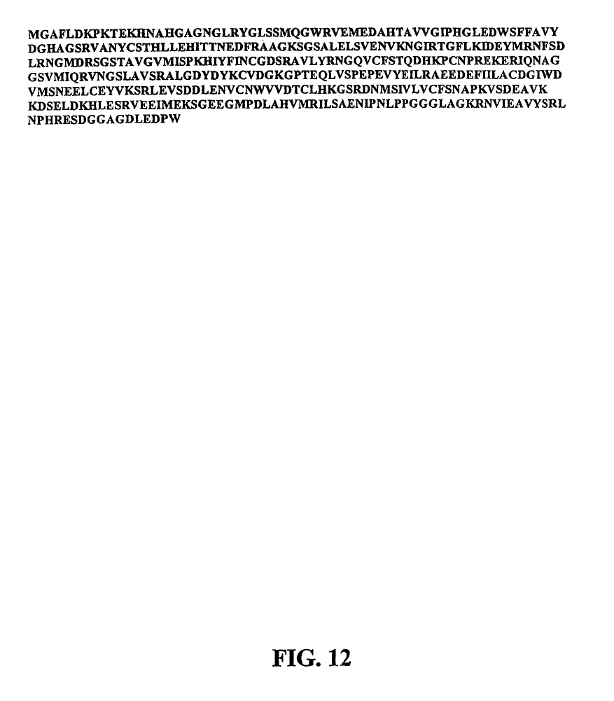

Human protein phosphatase 2C_{.}β (43 kD) was cloned and sequenced for the first time. The sequence of the DNA is recited in FIG. 11, and the corresponding amino acid sequence is recited in FIG. 12. The DNA sequence is SEQ ID NO 3. The corresponding amino acid sequence is SEQ ID NO 4, which differs in 19 amino acids from the rat PP2C_{.}β. Rat protein phosphatase 2Cβ_{.} (43 kD) is very similar in activity to PP2C_{.}α.

### Example 9

### Preparation of a HPMA Copolymer—Glucocerebrosidase Conjugate

Recombinant glucocerebrosidase is obtained from Genzyme.

The preparation of the HPMA copolymer-glucocerebrosidase conjugate is carried out using methods described in Example 2. The enzymatic activity of the glucocerebrosidase in the HPMA copolymer-glucocerebrosidase conjugate is measured using methods known in the art. See, for example, Pasmanik-Chor et al., 1997.

The HPMA copolymer-glucocerebrosidase conjugate is added to cell lines using methods similar to those described in Examples 3, 4 and 5. Visualization of the HPMA copolymer-glucocerebrosidase conjugate within the cell can be carried out using methods known in the art. An example of such a method is to obtain an antibody to glucocerebrosidase, label this antibody with a dye such as FITC (fluorescein isothiocyanate) and apply this labeled antibody to the treated cell lines, thereby visualizing the glucocerebrosidase by confocal microscopy or fluorescent microscopy, or alternatively by measurement of the biochemical activity of the enzyme. Delivery of glucocerebrosidase within the cell is demonstrated by this experiment.

The HPMA copolymer-glucocerebrosidase conjugate prepared as herein described is used to treat Gaucher Disease. Other storage diseases can also be treated by similar enzyme conjugates.

### Example 10

### Preparation of a HPMA Copolymer—Immunosuppressive Agent Conjugate

There are several immunosuppressive agents known in the art that are polypeptides. Such an immunosuppressive agent is obtained and preparation of the HPMA copolymer—immunosuppressive agent—conjugate is carried out using methods described in Example 2. The activity of the immunosuppressive agent in the conjugate is measured using methods known in the art.

The HPMA copolymer—immunosuppressive agent conjugate is added to cell lines using methods similar to those described in Examples 3, 4 and 5. Visualization of the conjugate within the cell can be carried out using methods known in the art. An example of such a method is to obtain an antibody, preferably a monoclonal antibody, to the immunosuppressive agent, label this antibody with a dye such as FITC (which liberates fluorescein) and apply this labeled antibody to the treated cell lines, thereby visualizing the immunosuppressive agent (as described in Example 5).

Delivery of an immunosuppressive agent within the cell is demonstrated by this experiment.

This conjugate prepared as herein described is used for immunosuppressive (anti-rejection) therapy, in particular in transplantation procedures. A very common transplantation procedure is corneal transplant and this conjugate is used to prevent rejection of the cornea following this procedure.

### Example 11

### Assessment of Functionality of Novel Polypeptides

A novel polypeptide, such as a polypeptide discovered (e.g. by translation from an EST) in the Human Genome Project, is obtained.

The preparation of the HPMA copolymer—novel polypeptide conjugate is carried out using methods described in Example 2.

The HPMA copolymer—novel polypeptide conjugate is added to cell lines using methods similar to those described as described in Examples 3, 4 and 5.

Visualization of the HPMA copolymer-polypeptide conjugate within the cell can be carried out using methods known in the art. An example of such a method is to construct a fusion protein as a tag (i.e. a known protein—novel polypeptide fusion protein, which is conjugated to the HPMA). An antibody, preferably a monoclonal antibody, that recognizes the known protein (the tag) in the fusion protein can then be used to visualize the conjugate within the cell. Other methods are described in Example 5.

Delivery of the novel polypeptide within the cell is demonstrated by this experiment.

Since intracellular delivery is achieved, assessment of functionality of the novel polypeptide (e.g. enzymatic activity) can be carried out.

### Example 12

### Preparation of other HPMA Copolymer—Polypeptide Conjugates

It is also possible to produce many other HPMA copolymer—polypeptide conjugates by using methods described in Example 2.

The HPMA copolymer—polypeptide conjugate is added to cell lines using methods similar to those described in Examples 3, 4 and 5. Visualization of the conjugate within the cell can be carried out using methods known in the art, for example, as described in Examples 5 and 10.

Delivery of various polypeptides within the cell is demonstrated by these experiments.

The HPMA copolymer—polypeptide conjugate prepared as herein described is used to treat many types of disorders.

The polypeptide may be, inter alia, an antibody, an intrabody, a toxin, or an enzyme.

### Example 13

Evaluation of the Body Distribution of HPMA Copolymer PP2C and Free PP2C in Mice Bearing B16F10.9 Melanoma

All animal experiments were conducted according to the United Kingdom Coordinating Committee on Cancer Research (UKCCCR) Guidelines.

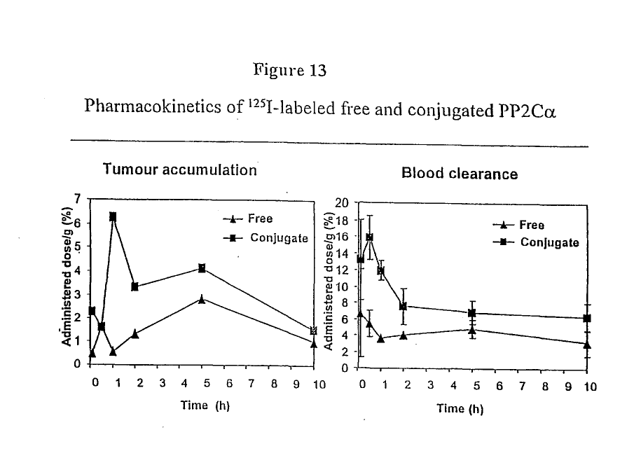

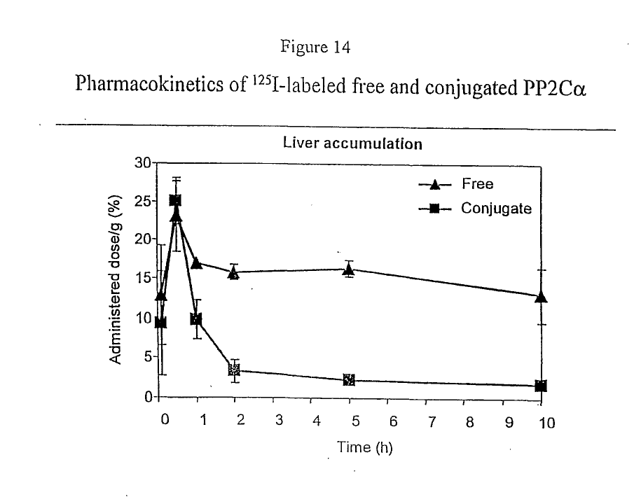

Male C57BL/6J mice were inoculated with 10⁵ viable B16F10 cells s.c. and the tumor was allowed to establish until the area was approximately 50-70 mm^{2.} The animals were injected i.v. with free or conjugated ¹²⁵I-labelled PP2C (5×10⁵ CPM/mouse) and animals culled at times up to 72 h. The main organs were dissected and the blood collected. The tumor, organs and blood samples were homogenized and read in a γ-counter. Results were calculated as % of administered dose/g. Body distribution of ¹²⁵I-labelled free and conjugated PP2Cα showed a 3-fold increase in tumor accumulation, 3-fold longer circulation time (FIG. 13) and significant 4-fold decrease in AUC of liver accumulation (FIG. 14).

### Example 14

Evaluation of Anti-Tumor Activity of HPMA Copolymer-PP2C Conjugate in Melanoma Model

Male C57Bl/65 mice were inoculated subcutaneously with 10⁵ viable B16F10.9 melanoma cells. The tumor was allowed to establish until its area was approximately 20-50 mm^{2.}

Animals were injected i.v. twice at day 1 and 5 with HPMA copolymer-PP2C conjugate. Several experiments were performed using rat PP2C and human PP2C conjugates at doses of 20 mg/Kg protein equivalent in saline. Control groups of mice were injected with 100 μl saline i.v. Each group consisted of 6 mice. Animals were weighed and the tumor size was measured daily. Animals were monitored daily for general health, weight loss and tumor progression. Throughout the experiment there was no weight loss, indicating that dose escalation and repeated dosage treatments are possible.

Mice were culled when the tumor reached or surpassed the size of 300 mm². At termination the animals were examined post-mortem and the tumors dissected and weighed.

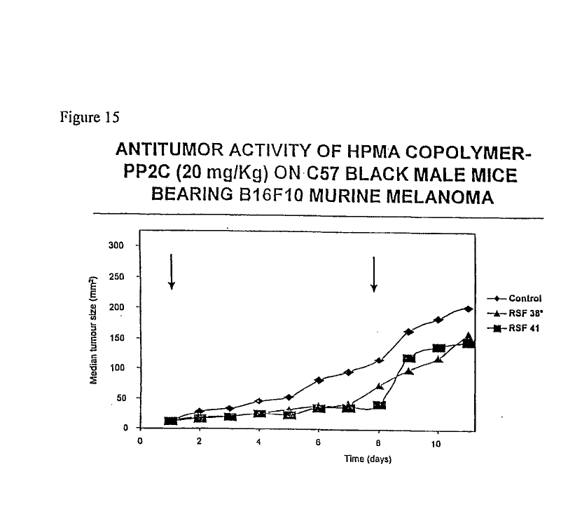

A significant decrease in tumor growth rate was observed after treatment with HPMA copolymer-PP2C compared to the control group (FIG. 15).

### Example 15

Dose Escalation Antitumor Activity Study of HPMA Copolymer—PP2C Conjugate in Melanoma Model

Male C57Bl/65 mice were inoculated subcutaneously with 10⁵ viable B16F10.9 melanoma cells. The tumor was allowed to establish until its area was approximately 20-50 mm^{2.}

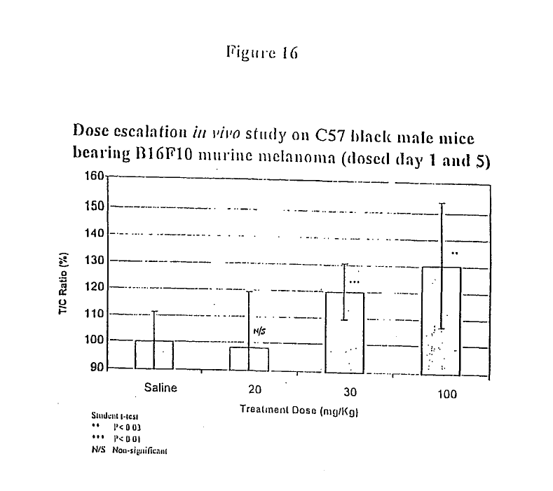

Animals were injected i.v. twice at day 1 and 5 with increasing doses (20-100 mg/Kg protein equiv.) HPMA copolymer-PP2C conjugate. Control groups of mice were injected i.v. with 100 μl saline. Each group consisted of 6 mice. Animals were weighed and the tumor size was measured daily. Increased survival was observed when treated with increased doses of HPMA copolymer-PP2C (T/C ratio of 130% at 100 mg/kg compared to the control group). Throughout the experiment there were neither toxic deaths nor animal weight loss even at the higher dose (100 mg/Kg) indicating that maximum tolerated dose (MTD) was not attained (FIG. 16).

### REFERENCES

Baykov et al., 1988, Analytical Biochemistry, 171:266-270.

Buolamwini, J. K., 2000, Curr. Pharm. Des., Mar., 6(4):379-392.

Cheng, A. T al., 2000, Journal of Biological Chemistry, August 16 edition.

Cohen, 1989, Ann. Review Biochem., 58:453-508.

Duncan, 1992, Anti-Cancer Drugs, 3: 175-210.

Duncan et al, 1987, Br. J. Cancer, 55:165-174.

Duncan et al, 1992, Anti-cancer drugs, 3:175-210.

Duncan et al., 1996, S. T. P. Pharma Sci., 6(4): 237-263.

Eden and Cedar, 1994, Curr. Opin. Genet. Dev., 4(2):255-9.

Gregoriadis, 1989, Targeting of drugs: Implications in medicine, in Drug Carrier Systems, Ed., Roerdink and Kroon, Horizons in Biochemistry and Biophysics, 9: 1-31.

Kataoka, 1997, Targetable polymeric drugs, In: Controlled Drug Delivery-Challenges and Strategies, Ed. Park, American Chemical Soc., U.S.A., 49-71.

Kato, S. et al, 1995, Arch. Biochem. Biophys., 318:387-393.

Kerr et al., 1998, 3^{rd} Int. Symp. Polymer Therap, London, U.K., 23.

Kopecek and Bazilova, 1973, Eur. Polymer J., 9:7-14.

Lau and Baylink, 1993, Crit. Rev. Oncol., 4(4);451-71.

Li et al, 1997, Science, 275:1943-1947.

Marley et al, 1998, Biochem. J., 320:801-806.

McGowan and Cohen, 1987, Eur. J. Biochem., 166:713-722.

Mutschler and Derendorf, 1995, Chemotherapy of Malignant Tumors, In: Drug Actions, Medpharm Scientific Pubs., Stuttgart, 595-612.

Nishikawa et al, 1995, FEBS Lett., 375:299-303.

Pasmanik-Chor et al., 1997,6 (6):997-895.

Saadat et al, 1994, Cancer Detection and Prevention, 18(2):115-122.

Scudiero, 1988, Cancer Research, 48:4827-4833.

Shiozaki and Russell, 1995a, The EMBO Journal., 14(3):492-502.

Shiozaki and Russell, 1995b, Nature, 378:739-743.

Shiozaki et al., 1994, Mol. Cell Biol.,14:3742-3751.

ten Bokkel Hunik et al., 1998, 3^{rd} Int. Symp. Polymer Therap, London, U.K., 12.

Terasawa et al., 1993, Arch. Biochem. Biophys., 307:342-349

Vasey et al., 1999, Clin. Cancer Research, 5:83-94

Wenk and Mieskes, 1995, Eur. J. Cell Biology, 68:377-386

Wenk, J. et al., 1992, FEBS Lett, 297:135-138

Yamauchi, T. et al., 2000, J. Biol. Chem., August 10 edition.

What is claimed is:

1. A complex molecule comprising a conjugate of a polymer capable of being taken up by a cell linked to a biologically active polypeptide, the conjugate capable of achieving intracellular delivery of the polypeptide while retaining its biological activity, with the proviso that the biologically active peptide is other than an antibody which binds to a cell surface marker or receptor.

2. The conjugate of claim 1, wherein the polymer is linked to the polypeptide by means of a direct chemical bond or a linker.

3. The conjugate of claim 2, wherein the linker is not degraded under physiological conditions.

4. The conjugate of claim 2, wherein the linker comprises a peptide.

5. The conjugate of claim 4, wherein the peptide is Gly-Gly.

6. The conjugate of claim 1, wherein the polymer is an N-alkyl acrylamide polymer.

7. The conjugate of claim 6, wherein the N-alkyl acrylamide polymer is a homopolymer.

8. The conjugate of claim 6, wherein the N-alkyl acrylamide polymer is a copolymer.

9. The conjugate of claim 8, wherein the copolymer comprises N-hydroxypropyl methacrylamide.

10. The conjugate of claim 1, wherein the polypeptide is selected from the group consisting of a therapeutic antibody, an intrabody, a toxin, or an enzyme.

11. The conjugate of claim 9, wherein the polypeptide is selected from the group consisting of a therapeutic antibody, an intra body, a toxin or an enzyme.

12. The conjugate of claim 9, wherein the polypeptide is an anti-tumor polypeptide.

13. The conjugate of claim 9, wherein the polypeptide is an anti-inflammatory polypeptide.

14. The conjugate of claim 9, wherein the polypeptide is a polypeptide for immunosuppressive therapy.

15. The conjugate of claim 9, wherein the polypeptide for immunosuppressive therapy is used in a transplantation procedure.

16. The conjugate of claim 9, wherein the polypeptide is a polypeptide for the treatment of a genetic disease.

17. The conjugate of claim 16, wherein the polypeptide is an enzyme.

18. The conjugate of claim 17, wherein the polypeptide is glucocerebrosidase.

19. The conjugate of claim 9, wherein the polypeptide is a polypeptide for therapy of an autoimmune disease.

20. The conjugate of claim 9, wherein the polypeptide is a polypeptide for preventing re-occlusion or re-stenosis.

21. A pharmaceutical composition comprising as an active ingredient a complex molecule comprising a conjugate of a polymer capable of being taken up by a cell linked to a biologically active polypeptide, the conjugate capable of achieving intracellular delivery of the polypeptide while retaining its biological activity, with the proviso that the biologically active peptide is other than an antibody which binds to a cell surface marker or receptor, together with a pharmaceutically acceptable carrier or diluent.

22. The composition of claim 21, wherein the polymer is linked to the polypeptide by means of a direct chemical bond or a linker.

23. The composition of claim 22, wherein the linker is not degraded under physiological conditions.

24. The composition of claim 22, wherein the linker comprises a peptide.

25. The composition of claim 24, wherein the peptide is Gly-Gly.

26. The composition of claim 21, wherein the polymer is an N-alkyl acrylamide polymer.

27. The composition of claim 26, wherein the N-alkyl acrylamide polymer is a homopolymer.

28. The composition of claim 26, wherein the N-alkyl acrylamide polymer is a copolymer.

29. The composition of claim 28, wherein the copolymer comprises N-hydroxypropyl methacrylamide.

30. The composition of claim 21, wherein the polypeptide is selected from the group consisting of a therapeutic antibody, an intrabody, a toxin, or an enzyme.

31. The composition of claim 29, wherein the polypeptide is selected from the group consisting of a therapeutic antibody, an intra body, a toxin or an enzyme.

32. The composition of claim 29, wherein the polypeptide is an anti-tumor polypeptide.

33. The composition of claim 29, wherein the polypeptide is an anti-inflammatory polypeptide.

34. The composition of claim 29, wherein the polypeptide is a polypeptide for immunosuppressive therapy.

35. The composition of claim 29, wherein the polypeptide for immunosuppressive therapy is used in a transplantation procedure.

36. The composition of claim 29, wherein the polypeptide is a polypeptide for the treatment of a genetic disease.

37. The composition of claim 36, wherein the polypeptide is an enzyme.

38. The composition of claim 37, wherein the polypeptide is glucocerebrosidase.

39. The composition of claim 29, wherein the polypeptide is a polypeptide for therapy of an autoimmune disease.

40. The composition of claim 29, wherein the polypeptide is a polypeptide for preventing re-occlusion or re-stenosis.

41. Use for the preparation of a medicament of a complex molecule comprising a conjugate of a polymer capable of being taken up by a cell linked to a biologically active polypeptide, the conjugate capable of achieving intracellular delivery of the polypeptide while retaining its biological activity, with the proviso that the biologically active peptide is other than an antibody which binds to a cell surface marker or receptor.

42. Use for the preparation of a medicament of a conjugate according to any one of claims 21-35.

43. Use according to claim 41, wherein the polypeptide is a polypeptide for the treatment of a genetic disease.

44. Use according to claim 43, wherein the polypeptide is an enzyme.

45. Use according to claim 44, wherein the polypeptide is glucocerebrosidase.

46. Use according to claim 45, for preparation of a medicament for the treatment of Gaucher's disease.

47. Use of the composition of claim 38, in the treatment of Gaucher's disease.

48 The complex molecule of claim 1, further comprising an internal protein localization signal.

49. The complex molecule of claim 1, which further comprises at least one additional drug or targetor moiety.

50. The complex molecule of claim 9, further comprising an internal protein localization signal.

51. The complex molecule of claim 9, further comprising at least one additional drug or targetor moiety.

52. A method of treating a subject suffering from a disorder which comprises administering to the subject a therapeutically effective amount of the composition of claim 21.

53. A method of treating a subject suffering from a disorder which comprises administering to the subject a therapeutically effective amount of the composition of claim 29.

54. A method of treating a symptom in a subject which comprises administering to the subject a therapeutically effective amount of the composition of claim 29.

55. A method which comprises contacting a eukaryotic cell with a composition comprising a carrier and a polymer capable of being taken up by a cell, said polymer being linked to a polypeptide, wherein the polypeptide enters the cell and exhibits an enzymatic activity within the cell.

56. A method of claim 70, wherein the enzymatic activity is glucocerebrosidase activity.
